(19) United States
(12) Patent Application Publication
(10) Pub. No.: US 2024/0226444 A1
(43) Pub. Date: Jul. 11, 2024

(54) MEDICAL SYRINGE

(71) Applicant: Covidien LP, Mansfield, MA (US)

(72) Inventor: Christian Lars Halvorsen, Minneapolis, MN (US)

(21) Appl. No.: 18/559,219

(22) PCT Filed: May 5, 2022
(86) PCT No.: PCT/US2022/027878

§ 371 Date: Nov. 6, 2023

Related U.S. Application Data
(60) Provisional application No. 63/185,034, filed on May 6, 2021.

(51) Int. Cl. A61M 5/315; A61M 5/31

(52) U.S. Cl. CPC A61M 5/31526; A61M 5/3135; A61M 5/31505; A61M 5/31571; A61M 5/31573; A61M 5/31595; A61M 2005/3101; A61M 2005/3104; A61M 2005/3121; A61M 2005/3142; A61M 2005/31508; A61M 2202/0007

(57) ABSTRACT

A medical syringe includes a ratchet for use during dispensing. The medical syringe, by virtue of the ratchet, enables repeated dispensing of a fixed amount of a relatively high-viscosity fluid to a target site within a patient. The medical syringe may be pre-filled with fluid and the fluid maybe sealed within the medical syringe. Accordingly, the medical syringe is configured to resist leaks and/or breaches that may occur as a result of manufacturing, sterilization, shipping, and/or storage.

### FIELD

The present technology is generally related to a medical syringe. For example, the medical syringe may be configured for dispensing a relatively high-viscosity fluid, as compared to water, to a target site within a patient. In this or another example, the medical syringe is configured to be pre-filled with fluid and seal the fluid within the medical syringe.

### BACKGROUND

Syringes may be used to deliver fluid to a target site within a patient. Commonly, a hypodermic needle may be attached to a tip of a syringe to inject a low-viscosity fluid (e.g., drug suspension) into a muscle (i.e., intramuscular injection) or a vein (i.e., intravenous injection), for example. Syringes may also be used to deliver relatively high-viscosity fluids to a target site within a patient for medical treatment of the patient. For example, a syringe may be used to deliver a high-viscosity fluid into a vein or other blood vessel.

In one such example, a syringe may be used to deliver an adhesive material into a vein via a catheter to permanently close a lower extremity vein (e.g., a lower extremity superficial truncal vein) for treating, for example, chronic venous insufficiency. The adhesive or other substance may be provided in a vial or other container, and the syringe is filled from the vial, such as immediately prior to or during the procedure. Conventionally, the syringe is coupled to a mechanical dispensing unit (e.g., an “adhesive gun”) to provide a mechanical advantage for repeatedly delivering desired amounts of the adhesive from the syringe. An example of a mechanical dispensing unit is described in U.S. Ser. No. 15/591,172, filed Jan. 7, 2015, now issued as U.S. Pat. No. 9,883,856.

### SUMMARY

In one aspect, the present disclosure generally relates to a medical syringe. The medical syringe comprises a barrel including a barrel body having proximal and distal ends and a longitudinal axis extending therebetween. The barrel body is configured to hold a volume of relatively high-viscosity fluid therein. A plunger includes a plunger body longitudinally slidable distally in the barrel body to dispense the relatively high-viscosity fluid from the barrel. A ratchet is configured to enable repeated dispensing of a fixed amount of the relatively high-viscosity fluid from the syringe to a target site within a patient. The ratchet includes a toothed rack extending along the plunger body and including a plurality of teeth spaced apart from one another along the plunger body, and a pawl connected to the barrel and engageable with the toothed rack.

In another aspect, the present disclosure generally relates to a pre-filled medical syringe. The pre-filled medical syringe comprises a barrel. The barrel includes a barrel body having proximal and distal ends and a longitudinal axis extending therebetween. The barrel body defines a longitudinal barrel passage extending through the proximal and distal ends and configured to slidably receive a plunger therein for dispensing fluid from the barrel body. A proximal closure cap is removably secured to the proximal end of the barrel body and configured to close and seal the barrel passage the proximal end thereof. A distal closure cap is removably secured to the distal end of the barrel body and configured to close and seal the barrel passage and the distal end thereof. A relatively high-viscosity medical grade fluid is pre-filled in the barrel body. The barrel is free from a plunger received in the barrel body.

In yet another aspect, the present disclosure generally relates to a medical syringe. The medical syringe comprises a barrel including a barrel body having proximal and distal ends and a longitudinal axis extending therebetween. The barrel body is configured to hold a volume of fluid therein. A plunger includes a plunger body having proximal and distal ends and a longitudinal axis extending therebetween. The distal end of the plunger body closes and seals the proximal end of the barrel body. The plunger body is releasably fixed longitudinally relative to the barrel body to inhibit the plunger from sliding longitudinally within to the barrel body when a longitudinal force is applied to the plunger. Fluid is pre-filled in the barrel body.

In another aspect, the present disclosure generally relates to a medical syringe. The medical syringe comprises a barrel including a barrel body having proximal and distal ends and a longitudinal axis extending therebetween. The barrel body is configured to hold a volume of relatively high-viscosity fluid therein. A proximal closure cap is removably secured to the proximal end of the barrel body and configured to close and seal the barrel passage the proximal end thereof. A distal closure cap is removably secured to the distal end of the barrel body and configured to close and seal the barrel passage and the distal end thereof. A relatively high-viscosity medical grade fluid is pre-filled in the barrel body. A plunger includes a plunger body configured to be longitudinally slidable distally in the barrel body to dispense the relatively high-viscosity fluid from the barrel. A ratchet is configured to enable repeated dispensing of a fixed amount of the relatively high-viscosity fluid from the syringe to a target site within a patient. The ratchet includes a toothed rack extending along the plunger body and including a plurality of teeth spaced apart from one another along the plunger body, and a pawl connected to the barrel and engageable with the toothed rack.

In another aspect, the present disclosure generally relates to a medical syringe. The medical syringe comprises a barrel including a barrel body having proximal and distal ends and a longitudinal axis extending therebetween. The barrel body is configured to hold a volume of relatively high-viscosity fluid therein. A plunger includes a plunger body longitudinally slidable distally in the barrel body to dispense the relatively high-viscosity fluid from the barrel. The plunger body is releasably fixed longitudinally relative to the barrel body to inhibit the plunger from sliding longitudinally within to the barrel body when a longitudinal force is applied to the plunger. A ratchet is configured to enable repeated dispensing of a fixed amount of the relatively high-viscosity fluid from the syringe to a target site within a patient. The ratchet including a toothed rack extending along the plunger body and including a plurality of teeth spaced apart from one another along the plunger body, and a pawl connected to the barrel and engageable with the toothed rack. A fluid is pre-filled in the barrel body.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a perspective of a medical syringe including a ratchet constructed according to the teachings of the present disclosure.

FIG. 2 is an enlarged front elevation of a plunger of the medical syringe, a toothed rack of the ratchet being connected to the plunger.

FIG. 3 is an enlarged perspective of a pawl of the ratchet.

FIG. 4A is a top plan view of the pawl.

FIG. 4B is a cross section of the pawl taken through the line 4B-4B in FIG. 4A.

FIG. 5 is an enlarged detail of the plunger as indicated in FIG. 2.

FIG. 6 is an enlarged detail of the syringe as indicated in FIG. 1.

FIG. 7 is a front elevation of another embodiment of a medical syringe including a ratchet.

FIG. 8 is an enlarged, fragmentary perspective of the medical syringe.

FIG. 9 is a front elevation of a medical syringe suitable for being pre-filled with fluid.

FIG. 10 is similar to FIG. 9, but with a proximal closure cap shown removed from a barrel of the medical syringe.

FIG. 11 is a similar to FIG. 10, but with a plunger shown inserted into the barrel.

FIG. 12 is a front elevation of a barrel of another embodiment of a medical syringe suitable for being pre-filled with fluid.

FIG. 13 is an enlarged, fragmentary perspective of a proximal end of the barrel.

FIG. 14 is an enlarged, fragmentary perspective of a distal end of a plunger of the medical syringe.

FIG. 15 is an enlarged, fragmentary front elevation of the medical syringe showing the plunger releasably fixed in a longitudinal position on the barrel.

FIG. 16 is similar to FIG. 15, but with the plunger released from the barrel and capable of sliding longitudinally within the barrel.

FIG. 17 is an enlarged, fragmentary perspective of another embodiment of a medical syringe suitable for being pre-filled with fluid.

FIG. 18 is an enlarged, fragmentary front elevation of a proximal end of a barrel of the medical syringe.

FIG. 19 is an enlarged, fragmentary front elevation of a distal end of a plunger of the medical syringe.

FIG. 20 is an enlarged perspective of a pin of the medical syringe.

FIG. 21 is a perspective of another embodiment of a medical syringe suitable for being pre-filled with fluid.

FIG. 22 is an enlarged detail of the medical syringe as indicated in FIG. 21.

FIG. 23 is an enlarged perspective of a cage of the medical syringe.

### DETAILED DESCRIPTION

The present description relates to a medical syringe. In one embodiment, the medical syringe includes a ratchet for use during dispensing. The medical syringe, by virtue of the ratchet, is suitably configured to enable repeated dispensing of a fixed amount of a relatively high-viscosity fluid to a target site within a patient. As used herein, “relatively high-viscosity fluid” is as compared to water. In this or a separate embodiment, the medical syringe is configured to be pre-filled with fluid and seal the fluid within the medical syringe. Accordingly, the medical syringe is configured to resist leaks and/or breaches that may occur as a result of manufacturing, sterilization, shipping, and/or storage.

### Medical Syringe Including Ratchet

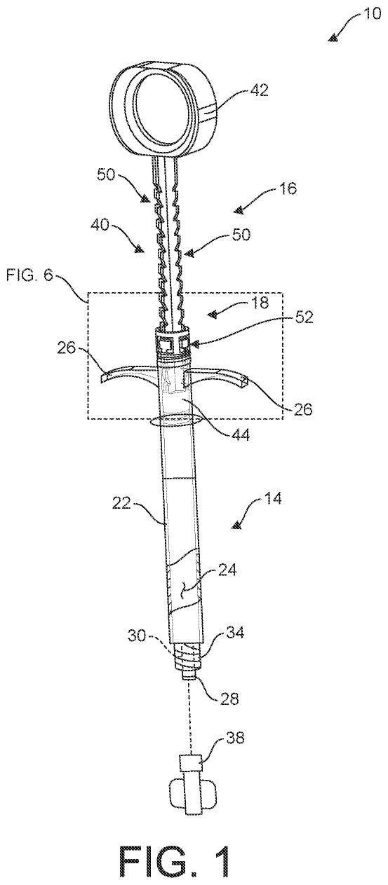

Referring to FIG. 1, an illustrated example of a medical syringe with a ratchet for use during dispensing is generally indicated at reference numeral 10. The medical syringe 10 includes a barrel 14, a plunger 16 slidably coupled in the barrel, and a ratchet 18, each of which is indicated generally. As disclosed above, the medical syringe 10 is configured to enable repeated dispensing of a fixed amount of a relatively high-viscosity fluid to a target site within a patient. Broadly, the syringe may be referred to as an indexing-type syringe. The syringe 10 is configured to enable a practitioner to dispense the fluid manually without a separate device providing a mechanical advantage. In one example, the relatively high-viscosity fluid is a medical grade adhesive suitable for closing a lower extremity vein (e.g., a lower extremity superficial truncal vein) for treating, for example, chronic venous insufficiency. One such adhesive includes n-butyl-2-cyanoacrylate. Another adhesive includes VenaSeal adhesive, which is commercially available from Medtronic.

Referring still to FIG. 1, the barrel 14 of the syringe 10 includes a barrel body 22, which may be generally cylindrical, having opposite proximal and distal ends and a length extending therebetween. The barrel body 22 defines a longitudinal body passage 24 extending through the proximal end toward the distal end. The body passage 24 is configured to hold a desired amount of the fluid to be dispensed and to receive the plunger 16 therein. A barrel flange 26 extends laterally outward from the barrel body 22 adjacent the proximal end thereof. The barrel flange 26 is configured to be engaged with fingers of the practitioner to aid in dispensing. A tip 28 of the barrel 14 extending distally from the distal end of the barrel body 22 defines a tip passage 30 in fluid communication with the body passage. The tip 28 may include an adaptor 34 (e.g., a luer lock or other adapter) configured to mechanically and fluidly couple to a catheter that is insertable into the patient, such as a vein or other blood vessel. A tip cap 38 may be removably coupled to the tip 28. The barrel body 22, barrel flange 26, and tip 28 may be integrally formed as a one-piece component, and may be formed from plastic or other suitable material. As explained below, the illustrated barrel 14 also includes a ratchet component of the ratchet 18.

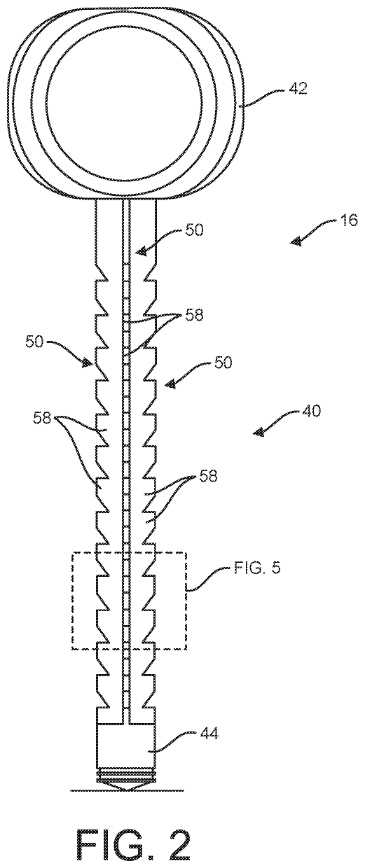

Referring to FIGS. 1 and 2, the plunger 16 of the syringe 10 includes a plunger body generally indicated at 40, having opposite proximal and distal ends and a length extending therebetween. A plunger flange 42 extends laterally outward from the plunger body 40 adjacent the proximal end thereof. The illustrated plunger flange 42 includes a ring although it may be of other shapes. The plunger flange 42 is configured to be engaged by a thumb of the practitioner that is on the same hand as the fingers engaging the barrel flange 26, as is generally understood by those in the art. A plunger seal 44 is coupled to the plunger body 40, such as adjacent the distal end of the plunger body. The seal 44 and the plunger body 40 are configured for longitudinally slidable insertion and movement in the body passage 24 of the barrel body 22. The seal 44 is configured to slidably engage and seal against the barrel body 22 such that moving the plunger 16 distally in the barrel body 22 (e.g., depressing the plunger) dispenses the fluid in the body passage 24 by forcing the fluid into and through the tip passage 30 to outside the syringe, such as into a catheter or other device, as would be generally understood to those in the art. The plunger body 40 and flange 42 may be integrally formed as a one-piece component, such as from plastic or other suitable material. The plunger seal 44 may be formed from rubber or other resiliently compressible material and may be overmolded on the plunger body 40 or connected thereto in other ways, such as adhesive or another mechanical connection. As explained below, the illustrated plunger 16 includes a ratchet component of the ratchet 18.

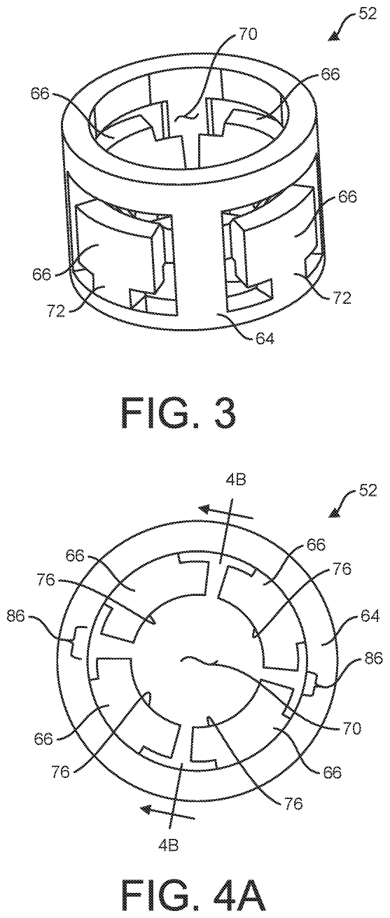

Referring to FIGS. 3-6, the ratchet 18 includes first and second mateable ratchet components which cooperate with one another (FIGS. 1 and 6) to enable repeated dispensing of a fixed amount of a relatively high-viscosity fluid to a target site within a patient. As briefly disclosed above, the barrel 14 includes one of the ratchet components and the plunger 16 includes the other ratchet component. In the illustrated embodiment, the first ratchet component comprises at least one toothed rack 50, and the second ratchet component comprises at least one pawl (or latch) 52 configured to engage the at least one toothed rack. In the illustrated embodiment, the toothed rack 50 is part of the plunger 16 and the pawl 52 is part of the barrel 14, with the understanding that the components may be reversed in other embodiments.

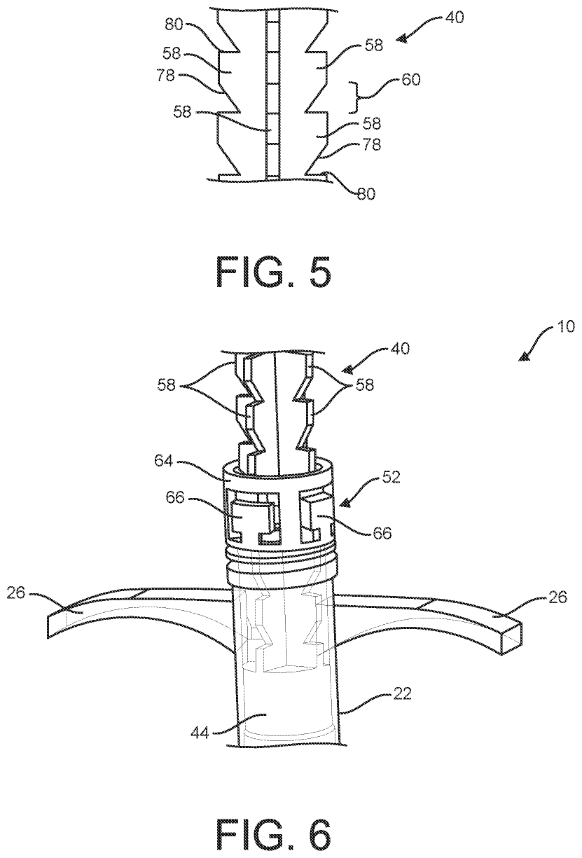

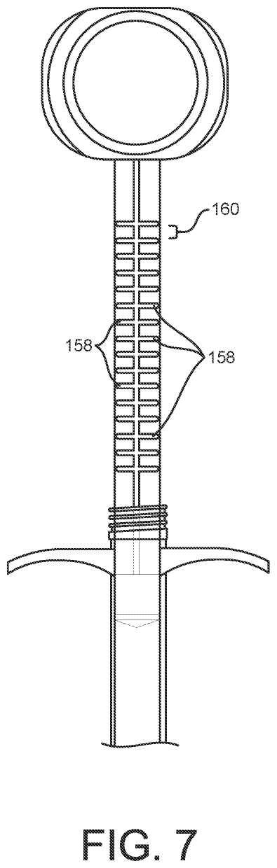

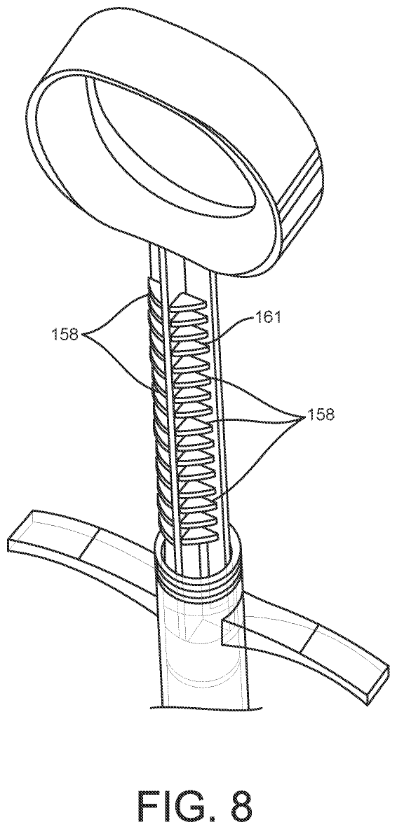

Referring to FIGS. 2 and 5, the toothed rack 50 includes more than one toothed rack (e.g., four racks, each indicated by reference numeral 50) running along the length of the plunger body 40 and spaced apart from one another around a longitudinal axis of the plunger body. Each toothed rack 50 includes a plurality of teeth 58 spaced apart from one another along the length of the plunger body 40. As shown in FIG. 5, tooth spaces 60 are defined between adjacent ones of the teeth 58 and are configured to receive the at least one pawl 52. In the illustrated embodiment, the toothed rack 50 is integrally formed with the plunger body 40, although it may be formed separate and subsequently secured thereto, such as by adhesive or other mechanical fastening ways. In one example, the teeth 58 and tooth spaces 60 are formed in longitudinal fins or ribs of the plunger body 40. In another example, such as shown in FIGS. 7-8, the teeth 158 and tooth spaces 160 may be disposed between adjacent longitudinal ribs 161 or at other locations.

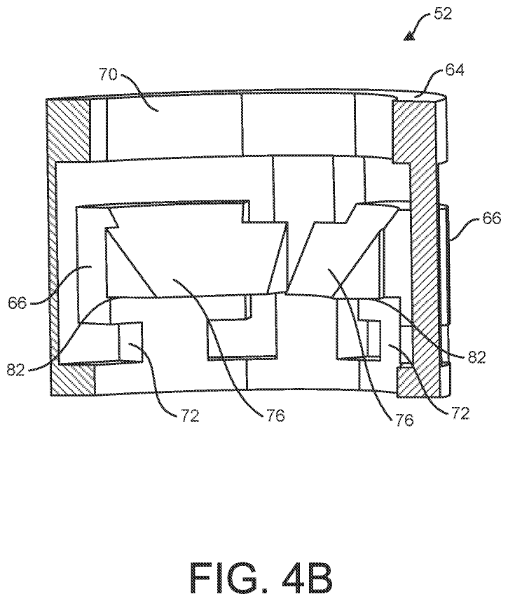

Referring to FIGS. 3 and 4, the pawl 52 includes a pawl body 64, and at least one pawl arm 66 connected to the pawl body. The pawl body 64 and the pawl arm 66 may be integrally formed as a one-piece component. The illustrated pawl 52 is disposed connected to the proximal end of the barrel body 22 and disposed outside the barrel body. The pawl 52 and the barrel body 22 may be integrally formed as a one-piece component or the components may be formed separate and connected to one another such as by adhesive or other mechanical fastening ways. In other embodiments, the pawl 52 may be disposed inside the barrel body 22. In yet another embodiment, the pawl body may be omitted, and the pawl 52 may include the at least one pawl arm 66 directed connected to (e.g., integrally formed with) the barrel body 22.

The illustrated pawl body 64 is generally cylindrical, similar to the shape of the barrel body 22, and defining a longitudinal through opening 70 aligned with the longitudinal axis of the barrel body and sized and shaped to receive the plunger body 40 therethrough. The pawl body 64 may have other shapes. The illustrated pawl 52 includes a plurality of a pawl arms 66 (e.g., four pawl arms) spaced apart from one another around the pawl body 64. The pawl arms 66 are connected to the pawl body 64 by respective living hinges 72 and are individually and resiliently deflectable in a radially outward direction relative to the pawl body 64 about the respective living hinges. Each pawl arm 66 include a pawl tooth 76 (e.g., an undercut) at an end thereof extending radially inward. The pawl teeth 76 are configured for incremental reception in the tooth spaces 60 of an associated one of the toothed racks 50 as the plunger 16 is depressed during use, as explained below.

In use, the ratchet 18 enables the plunger 16 to be depressed incrementally at discrete intervals based on uniform distances between the tooth spaces 60 to selectively dispense fixed amounts of fluid from the syringe 10. In an initial position, each pawl tooth 76 is in its biased configuration and received in an initial tooth space 60 of the corresponding rack 50. As the plunger 16 is depressed in the barrel 14, each pawl tooth 76 engages and rides along a ramp portion 78 (e.g., lower portion) of an adjacent one of the rack teeth 58 of the corresponding toothed rack 50. As it rides along the rack tooth 58, the pawl tooth 76 resiliently deflects radially outward to its deflected configuration, providing clearance for the rack tooth to slide passed the pawl tooth 76. Upon the rack tooth 58 clearing the pawl tooth 76, the pawl tooth rebounds to its biased configuration and enters the next sequential tooth space 60. Each time the pawl tooth 76 enters a sequential tooth space 60, the plunger 16 has moved a fixed, uniform distance within the barrel 14, and correspondingly dispensing the fixed amount of fluid from the syringe.

In the illustrated embodiment, upper surfaces 80 (FIG. 5) of the rack teeth 58 and opposing lower surfaces 82 (FIG. 4B) of the pawl teeth 76 are generally orthogonal to the length of the plunger body 40 and are engageable with one another to inhibit the pawl teeth from withdrawing from the tooth space 60 in which it is received, thereby inhibiting the plunger 16 from withdrawing (e.g., sliding proximally) relative to the barrel 14. In general, this configuration is an anti-backoff mechanism to inhibit backing out of the plunger 16, which is desirable when dispensing high-viscous material. To selectively withdraw the plunger 16, the practitioner rotates the plunger about its axis relative to the barrel to align toothed racks 50 with respective circumferential gaps 86 (FIG. 4A) between adjacent pawl arms 66 (and pawl teeth 76). The circumferential gaps 86 are sized and shaped to enable the toothed racks 50 to slide therethrough, thereby bypassing the pawl teeth 76 and enabling the plunger 16 to be withdrawn. The plunger 16 may be capable of being withdrawn in other ways, or may not be capable of being withdrawn.

### Medical Syringe Configured to be Pre-Filled and Sealed

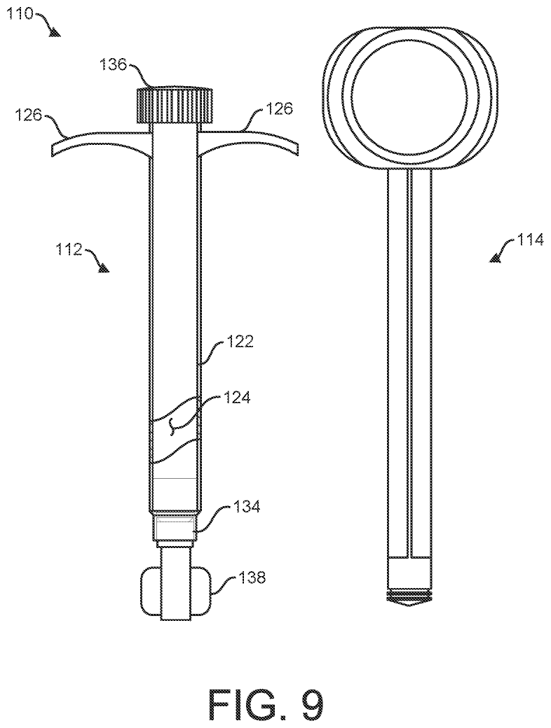

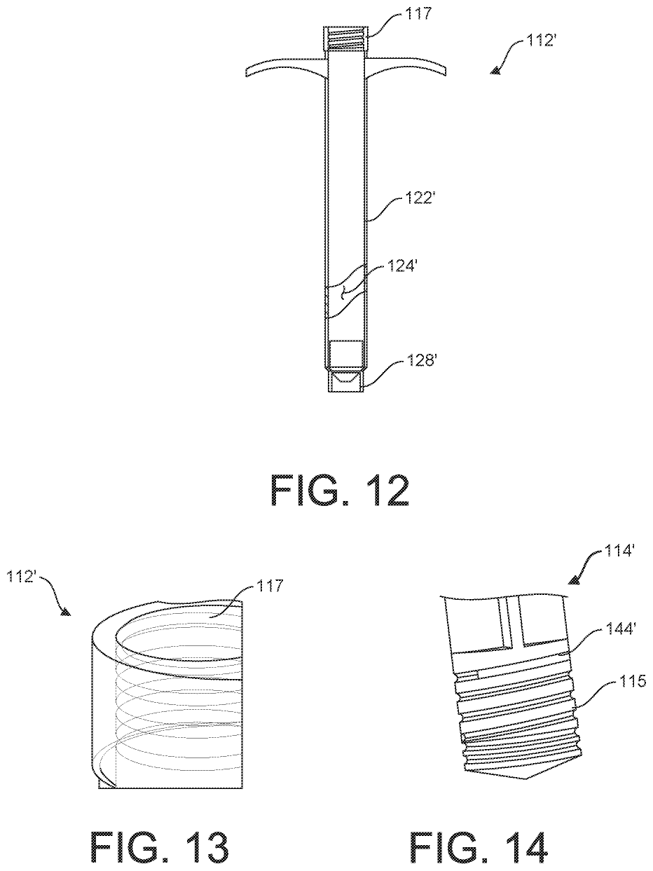

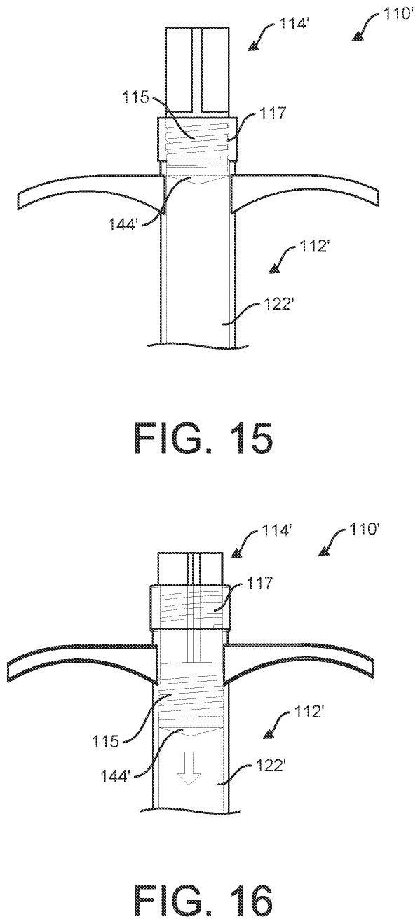

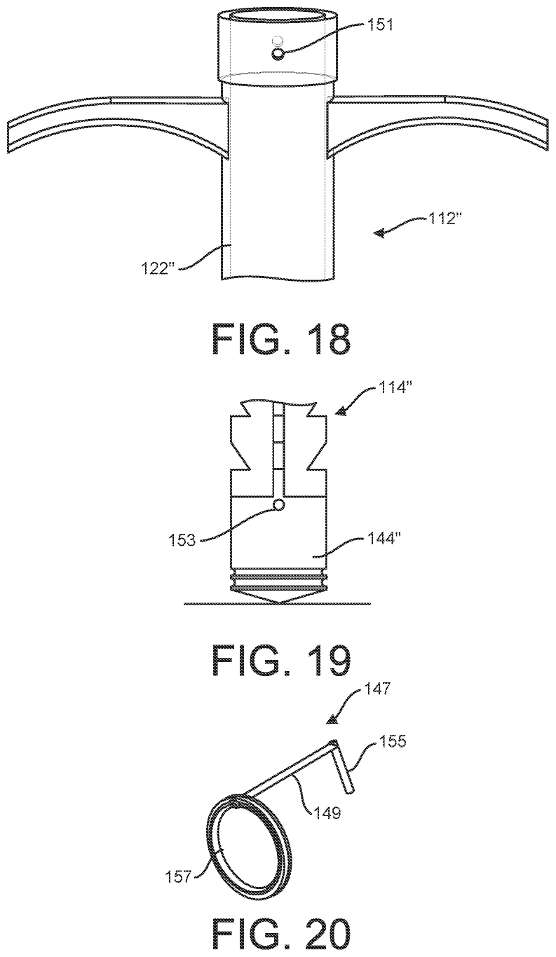

Referring to FIGS. 9-11, one embodiment of a medical syringe configured to be pre-filled and sealed is generally indicated at reference numeral 110. In FIGS. 12-23, additional embodiments of the medical syringe are indicated by corresponding reference numeral plus a consecutive number of prime symbols. These embodiments are described below, with a focus of identifying differences between the embodiments. It is understood that one or more of the features described herein below may be incorporated into the medical syringe including a ratchet, as described above. It is also understood that one or more of the features described herein below may be combined with one another in the same medical syringe.

Referring still to FIGS. 9-11, the medical syringe 110 includes a barrel 112 and a plunger 114 receivable in the barrel to dispense fluid disposed within the barrel. The plunger 114 may be a conventional plunger or may be designed and constructed according to the teachings of the syringe 10 with a ratchet, described above. Other than the features described below, the barrel may be a conventional barrel or may be designed and constructed according to the teachings of the syringe with a ratchet described above. In general, the barrel 110 includes a barrel body 122, which may be generally cylindrical, having opposite proximal and distal ends and a length extending therebetween. The barrel body 122 defines a longitudinal body passage 124 extending through the proximal end toward the distal end. The body passage 124 is configured to hold a desired amount of the fluid to be dispensed and to receive the plunger 116 therein. A barrel flange 126 extends laterally outward from the barrel body 22 adjacent the proximal end thereof. The barrel flange 126 is configured to be engaged with fingers of the practitioner to aid in dispensing. A tip or hub (hidden in drawings) of the barrel 14 extending distally from the distal end of the barrel body 22 defines a tip passage (hidden in drawings) in fluid communication with the body passage. The tip 28 may include an adaptor 134 (e.g., a luer lock or other adapter) configured to mechanically and fluidly couple to a catheter that is insertable into the patient, such as a vein or other blood vessel. The barrel body 122, barrel flange 126, and tip 128 may be integrally formed as a one-piece component, and may be formed from plastic or other suitable material.

In addition to the above features, the illustrated barrel 112 includes a proximal closure cap 136 removably attached to the proximal end of the barrel body 122 to close the proximal end of the body passage 124, and a distal closure cap 138 removably attached to the tip 128 to close the distal end of the tip passage 130. The illustrated proximal closure cap 136 is internally threaded and threaded on external threads 139 at the proximal end of the barrel body 122. The distal closure cap 138 may be coupled to the adaptor 134 of the tip 128. For example, the distal closure cap 138 may include a luer lock coupling configured to mate with a luer lock adaptor of the tip 128. Each of the caps 136, 138 may be removably secured to the barrel body 122 in other ways. Each cap 136, 138 may include a borosilicate glass and polytetrafluoroethylene (PTFE) liner, which is suitable for use with an adhesive fluid that includes a cyanoacrylate, such as, for example, an n-butyl-cyanoacrylate or an n-butyl-2-cyanoacrylate.

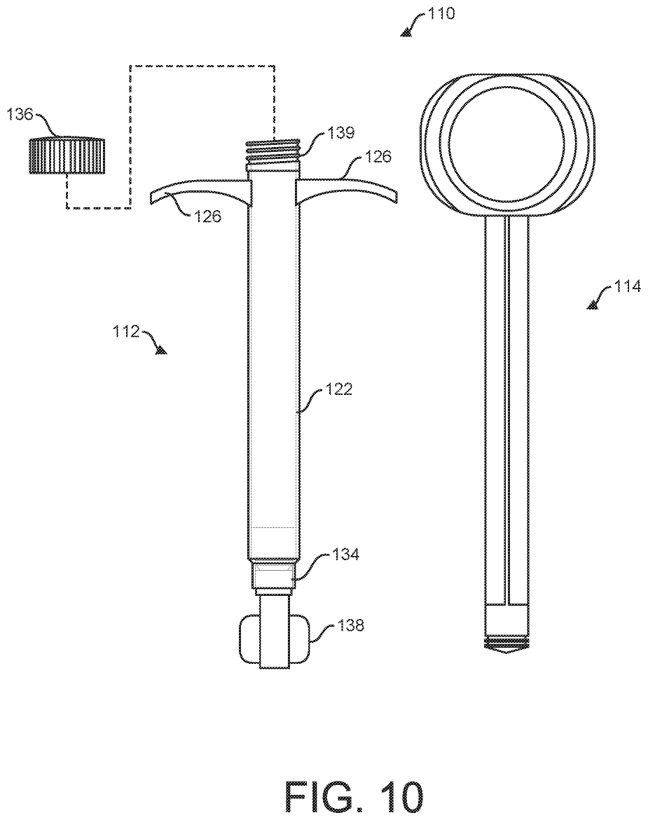

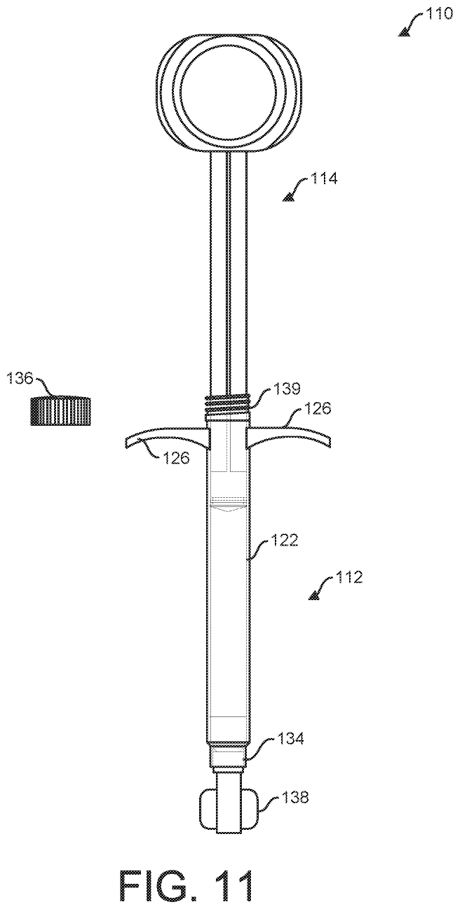

The caps 136, 138 enable the barrel 112 of the syringe 110 to be pre-filled with fluid (e.g., an adhesive, such as an cyanoacrylate adhesive fluid) before being packaged and shipped to a destination for use. In this embodiment, the plunger 114 may be shipped with the barrel 112 in the same package as a kit, or in a separate package. Prior to immediate use, the proximal closure cap 136 may be removed (FIG. 10) and the plunger 114 may be inserted through the proximal end into the barrel passage 124 (FIG. 11). The distal closure cap 138 is also removed immediately prior to use to open the tip passage 130. A catheter or other device can be attached to the tip 128.

Referring to FIGS. 12-16, another example of a medical syringe configured to be pre-filed is generally indicated at 110′. This medical syringe 110′ is similar to the prior medical syringe 110, with differences described below. Like components are indicated by corresponding reference numerals including one prime symbol. The medical syringe 110′ generally includes a barrel 112′ and a plunger 114′. Although not illustrated, the medical syringe 110′ may include the distal closure cap 138 removably secured to the tip 128′. Unlike the first medical syringe 110, however, present medical syringe 110′ does not include the proximal closure cap 136. Instead, the plunger 114′ of the medical syringe 110′ is releasably securable to the barrel at the proximal end of the barrel body 122′ to selectively close the proximal end of the barrel body and inhibit the plunger from inadvertently moving relative to the barrel (e.g., inhibit depressing or withdrawing of the plunger via a longitudinal force applied to the plunger). In particular, the plunger 114′ includes a plunger coupling 115 configured to mate with a barrel coupling 117 to inhibit sliding movement of the plunger relative to the barrel, thus sealing the fluid in the barrel passage 124′. In the illustrated embodiment, the plunger coupling 115 comprises threads (e.g., external threads) configured to threadably couple with threads (e.g., internal threads) of the barrel coupling 117. In the illustrated embodiment, the plunger threads 115 are formed on the plunger seal 144′, although they may be at other locations on the plunger 114′. The plunger coupling 115 may be threaded through the barrel coupling 117 to be slidably received in the barrel passage 124′ and capable of being depressed inside the barrel to dispense the fluid.

As can be understood, the barrel 112′ of the syringe 110′ is capable of being pre-filled at a location remote from its destination and then the plunger 114′ can be coupled to the proximal end of the barrel body 122′ to close and seal the proximal end of the barrel passage 124′. Prior to immediate use, the plunger 114′ may be rotated relative to the barrel 112′ to thread the plunger coupling through the barrel coupling to release the plunger and enable it to be slidably moved within the barrel. The distal closure cap 138 is also removed immediately prior to use to open the tip passage. A catheter or other device can be attached to the tip 128′.

The plunger 114′ may be selectively and releasably fastened or coupled to the barrel 116′ in other ways to close the proximal end of the barrel passage 124′.

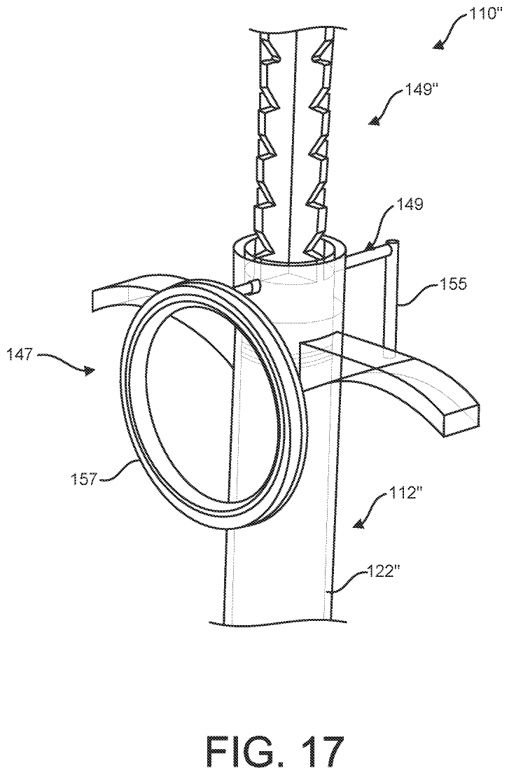

For example, referring to FIGS. 17-20, another example of the medical syringe 110″. This medical syringe 110″ is similar to the prior medical syringe 110′, with differences described below. Like components are indicated by corresponding reference numerals including two prime symbols. The medical syringe 110″ generally includes a barrel 112″ and a plunger 114″. Although not illustrated, the medical syringe 110″ may include the distal closure cap 138 removably secured to the tip 128. Instead of the threaded couplings 115, 117, the present medical syringe 110″ includes a selectively removable pin, generally indicated at reference numeral 147 (broadly, a plunger lock), including a pin body 149 extending through aligned openings 151, 153 in the barrel 112″ (e.g., barrel body 122″) and the plunger 114″. The aligned openings 151, 153 may be adjacent the proximal end of the barrel body 122″ and the distal end (or seal 144″) of the plunger 114″, respectively. The removable pin 147 may include a stop 155 at an end of the pin body 149 which inhibits the pin from being inadvertently removed from the openings 151, 153. The stop 155 may be removed from the pin body 149 (e.g., connected to the pin body by a frangible connection) or otherwise configured to enable the pin to be removable. A handle 157 at the other end of the pin body 149 is used to remove the pin 147. Once the pin 147 is removed, the plunger 114″ can be depressed and the fluid dispensed from the syringe 110″. The pin may be of other configurations.

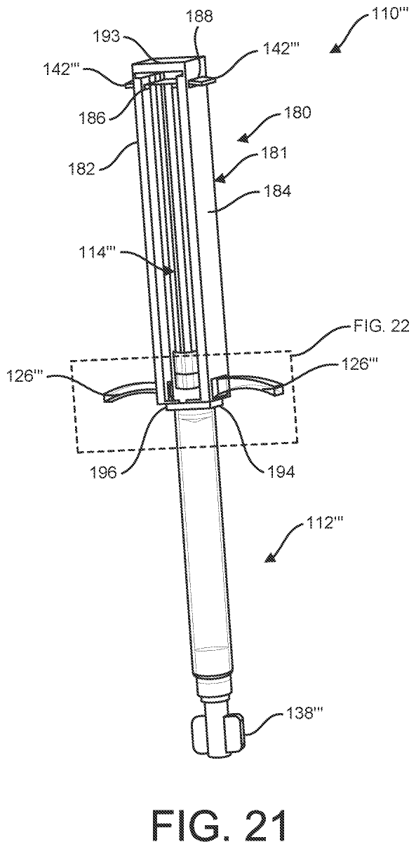

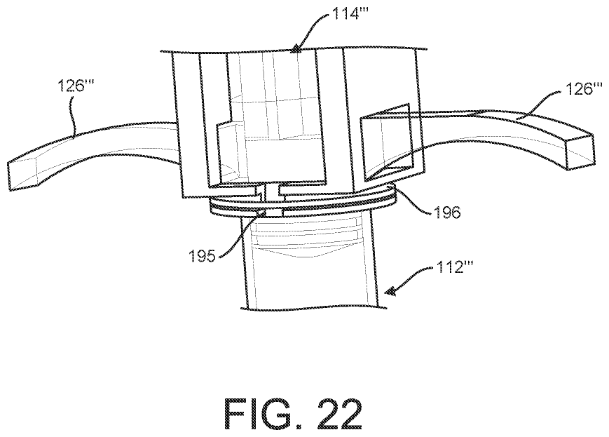

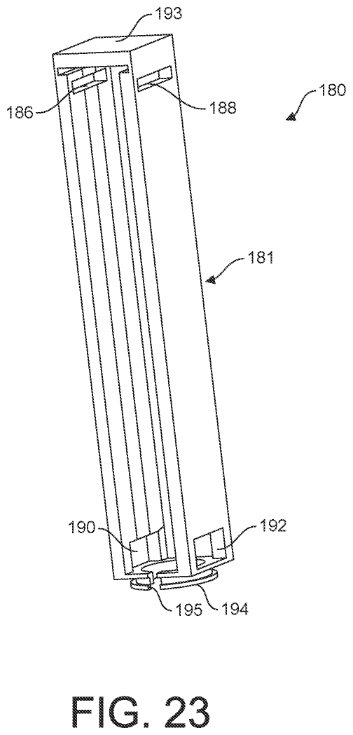

In another example, referring to FIGS. 21-23, a medical syringe 110′ is similar to the prior medical syringe 110′, with differences described below. Like components are indicated by corresponding reference numerals including three prime symbols. The medical syringe 110′″ generally includes a barrel 112′″ and a plunger 114′″. The medical syringe 110′″ may include the distal closure cap 138′″ removably secured to the tip. Instead of the threaded couplings 115, 117, the present medical syringe 110″ includes a removable plunger lock, generally indicated at reference numeral 180, configured to inhibit the plunger from being depressed without first removing the lock. The illustrated plunger lock 180 includes a cage, generally indicated at reference numeral 181. The cage 181 includes opposing and longitudinally extending sidewalls 182, 184 having proximal and distal ends. The side walls 182, 184 define opposing plunger-flange openings 186, 188, respectively, receiving the plunger flange 142′″, and opposing barrel-flange openings 190, 192, respectively, receiving the barrel flange 126′. A proximal end 193 of the cage 181 is closed and extending between the proximal ends of the sidewalls 182, 184. As shown in FIGS. 22 and 23, a distal end 194 of the cage 181 include slits 195 (one being shown) to enable the sidewalls 182, 184 to move away from each other at the distal end (or splay) to enable the cage to be received on the plunger 114′″ and barrel 112′″, as shown in FIG. 21. After placement of the cage 181 on the syringe 110′″, a tie or band 196 may be secured around the distal end of the cage to inhibit the distal end 194 from openings, thereby securing the cage to the syringe 110′″. Other ways of securing the distal ends of the sidewalls 182, 184 to one another may be employed.

As installed, the plunger lock 180 inhibits the plunger 114′″ from being depressed, thereby enabling the syringe to be pre-filled and shipped to its destination. At the destination and prior to use, the tie 196 is removed (e.g., cut). The cage 181 is then splayed open by moving the sidewalls 182, 184 away from one another at the distal end 194. This removes the barrel and plunger flanges 126′, 142′″, respectively, from the corresponding openings 186, 188 and 190, 192. The sidewalls 182, 184 may generally deflect adjacent the proximal end 193 (e.g., at a living hinge junction between the proximal end and the sidewalls 182, 184).

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

1. A medical syringe comprising:
a barrel including a barrel body having proximal and distal ends and a longitudinal axis extending therebetween, wherein the barrel body is configured to hold a volume of relatively high-viscosity fluid therein;
a plunger including a plunger body longitudinally slidable distally in the barrel body to dispense the relatively high-viscosity fluid from the barrel; and
a ratchet configured to enable repeated dispensing of a fixed amount of the relatively high-viscosity fluid from the syringe to a target site within a patient, the ratchet including
a toothed rack extending along the plunger body and including a plurality of teeth spaced apart from one another along the plunger body, and
a pawl connected to the barrel and engageable with the toothed rack.

2. The medical syringe set forth in claim 1, wherein the pawl includes a cylindrical pawl body and at least one pawl arm coupled to the pawl body, wherein the pawl body defines an opening aligned with the longitudinal axis of the barrel body and configured to receive the plunger and the toothed rack therethrough.

3. The medical syringe set forth in claim 2, wherein the at least one pawl arm is resiliently deflectable radially outward relative to the pawl body as it engages the teeth of the toothed rack during distal sliding movement of the plunger in the barrel body dispensing the relatively high-viscosity fluid from the barrel.

4. The medical syringe set forth in claim 2, wherein the pawl includes a plurality of pawl arms spaced apart circumferentially from one another about the pawl body.

5. The medical syringe set forth in claim 4, wherein the toothed rack includes a plurality of toothed racks, each toothed rack associated with one of the pawl arms.

6. The medical syringe set forth in claim 5, wherein each toothed rack defines tooth spaces between the teeth, wherein each pawl arm includes a pawl tooth configured for incremental reception in the tooth spaces of the associated the toothed racks as the plunger slides distally within the barrel body to dispense the relatively high-viscosity fluid from the barrel.

7. The medical syringe set forth in claim 6, further comprising an anti-backoff mechanism configured to inhibit each pawl tooth from withdrawing in a proximal direction from within the associated tooth space of the rack.

8. The medical syringe set forth in claim 1, further comprising an anti-backoff mechanism configured to inhibit the plunger from sliding proximally within the barrel body.

9. The medical syringe set forth in claim 1, further comprising medical grade adhesive fluid received in the barrel body, wherein the medical grade adhesive is suitable for closing a lower extremity vein.

10. The medical syringe set forth in claim 1, wherein the toothed rack is integrally formed with the plunger body.

11. The medical syringe set forth in claim 1, wherein the pawl is integrally formed with the barrel body.

12. A pre-filled medical syringe comprising:
a barrel including
a barrel body having proximal and distal ends and a longitudinal axis extending therebetween, wherein the barrel body defines a longitudinal barrel passage extending through the proximal and distal ends and configured to slidably receive a plunger therein for dispensing fluid from the barrel body,
a proximal closure cap removably secured to the proximal end of the barrel body and configured to close and seal the barrel passage the proximal end thereof, and
a distal closure cap removably secured to the distal end of the barrel body and configured to close and seal the barrel passage and the distal end thereof, and
a relatively high-viscosity medical grade fluid pre-filled in the barrel body,
wherein the barrel is free from a plunger received in the barrel body.

13. The pre-filled medical syringe set forth in claim 12, further comprising a plunger configured to be inserted into the barrel body through the proximal end thereof after removing the proximal closure cap.

14. The pre-filled medical syringe set forth in claim 13, wherein the proximal closure cap is threaded on the proximal end of the barrel body.

15. The pre-filled medical syringe set forth in claim 12, wherein the medical grade adhesive is suitable for closing a lower extremity vein.

16. A medical syringe comprising:
a barrel including a barrel body having proximal and distal ends and a longitudinal axis extending therebetween, wherein the barrel body is configured to hold a volume of fluid therein;
a plunger including a plunger body having proximal and distal ends and a longitudinal axis extending therebetween, wherein the distal end of the plunger body closes and seals the proximal end of the barrel body, the plunger body being releasably fixed longitudinally relative to the barrel body to inhibit the plunger from sliding longitudinally within to the barrel body when a longitudinal force is applied to the plunger; and
fluid pre-filled in the barrel body.

17. The medical syringe set forth in claim 16, wherein the plunger includes a plunger coupling coupled to the barrel to inhibit the plunger from sliding longitudinally within to the barrel body when a longitudinal force is applied to the plunger.

18. The medical syringe set forth in claim 17, further comprising a plunger lock releasably locking the plunger on the barrel.

19. (canceled)

20. (canceled)

21. The medical syringe set forth in claim 16, further comprising a distal closure cap removably secured to the distal end of the barrel body and configured to close and seal the barrel passage and the distal end thereof.

22. The medical syringe set forth in claim 18, wherein the plunger lock comprises a removable pin extending through aligned openings in the barrel and the plunger.
